(19) United States
(12) Patent Application Publication
(10) Pub. No.: US 2022/0160758 A1
(43) Pub. Date: May 26, 2022

(54) ANTIVIRAL, ANTIBACTERIAL AND/OR ANTI FUNGAL COMPOSITIONS, APPLICATIONS AND THERAPY

(71) Applicant: AVITAS BIO CORP., Inwood, NY (US)

(72) Inventors: Litvak Mark, Amir Gilboy (IL); HENRY VAL, Marlboro, NJ (US)

(21) Appl. No.: 17/535,911

(22) Filed: Nov. 26, 2021

Related U.S. Application Data
(60) Provisional application No. 63/118,594, filed on Nov. 25, 2020.

(51) Int. Cl. A61K 33/34; A61K 31/202; A61K 31/201

(52) U.S. Cl. CPC A61K 33/34; A61K 31/201; A61K 31/202

(57) ABSTRACT

Exemplary methods for inactivation of viruses, bacteria, and/or fungi includes contacting said viruses, bacteria, and/or fungi with an effective amount of a linear polyunsaturated C 20-24 fatty acid having 5-7 double bonds, a pharmaceutically acceptable salt of said acid, or a mixture thereof, and a copper sulfide incorporated therein. Local administration of the drug is preferred and effective in the treatment of lesions associated with infections of viruses, bacteria, and/or fungi. Exemplary pharmaceutical compositions for use in the present method are also provided.

### CROSS-REFERENCE TO RELATED APPLICATION(S)

This application relates to and claims priority from U.S. Patent Application Ser. No. 63/118,594 filed on Nov. 25, 2020, the entire disclosure of which is incorporated herein by reference.

### FIELD OF THE DISCLOSURE

The present disclosure relates to antiviral, antibacterial drugs, antifungal compositions such as drugs and to a method of inactivating a microorganism, such as a virus in an envelope, antibiotic-resistant bacteria, resistant fungal flora. In a composition aspect, the invention also relates to a pharmaceutical composition for use in the present method.

### BACKGROUND INFORMATION

In the past, viral infections have been largely resistant to antibiotic therapy. The herpes virus belongs to a class known as the “enveloped virus”, which refers to those DNA or RNA viruses that have a lipoprotein coat. Typically, the envelope of the virus is derived from the components of the host membrane under the influence of a viral protein. The class of enveloped viruses includes the herpes virus such as herpes simplex 1 and 2, myxovirus such as the influenza virus, paramyxovirus such as the virus that causes measles and mumps, and the respiratory syncytial virus responsible for croup, the corona virus that also causes the common cold, for example rubella virus and the virus that causes encephalitis and hemorrhagic fever.

Pathogenic bacteria contribute to other globally important diseases such as pneumonia, which can be caused by bacteria such as Streptococcus and Pseudomonas, and foodborne diseases, which can be caused by bacteria such as Shigella, Campylobacter and Salmonella. Pathogenic bacteria also cause infections such as tetanus, typhoid fever, diphtheria, syphilis, and leprosy. Pathogenic bacteria are also responsible for high infant mortality rates in developing countries.

Strep and Staphylococcus aureus are part of the normal skin microbiota and are usually found on healthy skin or around the nose. However, these species can potentially cause skin infections. They can also cause sepsis, pneumonia, or meningitis. These infections can become quite serious, triggering a systemic inflammatory response leading to massive vasodilation, shock and death.

Recent studies have shown that certain lipophilic compounds inhibit the replication of certain enveloped viruses in vitro. Various fatty acids can inhibit viral replication in bacteriophage. Without being bound by theory, at least two modes of fatty acid inhibition may be involved. The first mode involves inactivating the virus. Oleic acid, a C 18 monounsaturated fatty acid, was the most effective fatty acid tested for this property, but a C 18 acid having two double bonds was substantially inactive. The second mode is to suppress replication without destroying the virus, that is, antiviral or virustatic activity. This phenomenon is associated with the stage of the infection cycle at which fatty acid is added.

It has been shown that unsaturated fatty acids can inhibit viral replication of bacteriophage PR4 in vitro. The most effective acids were oleic acid and palmitoleic acid. Arachidonic acid (C 20 tetraene) was moderately effective but less effective than linolenic acid (C 18 triene).

The structure of the cell wall of bacteria and fungi, has a significant impact on how easily drug molecules can enter a fully formed bacterial or fungal cell. It is known that many membrane processes, such as transport or signaling, depend on the fluidity of membrane lipids, which, in turn, depends on the properties of fatty acid chains. Fatty acids in double membrane layers can exist in an ordered, rigid state or in a relatively disordered liquid state. The transition from rigid to liquid conformation and vice versa depends on temperature. This transition temperature depends on the length of the fatty acid chains and the degree of its unsaturation.

The membrane fluidity of bacteria and fungi can be (controlled) varied by using fatty acids with different numbers of double bonds and fatty acid chain lengths, which alters (e.g., shortens) their life cycle.

Berg, J., Tymoczko, J., Stryer, L. Biochemistry. W. H. Freeman and Company, New York, 2007 Kabara et al., Antimicrobial Agents and Chemotherapy, 2, 23-28 (1972) discloses that certain fatty acids inhibit the growth of gram-positive and gram-negative microorganisms. Some saturated fatty acids showed antibacterial activity, monounsaturated acids were more effective, and diene acids were even more active for C18 fatty acids. However, arachidonic acid was not inhibitory at the concentrations tested.

Sands et al., Antimicrobial Agents and Chemotherapy, 15, 67-73 (1979) discloses the in vitro antiviral activity of unsaturated C 14-20 alcohols having 1-4 double bonds, the most active of which is gamma-linolenyl alcohol (6, 9, 12-octadecatrien-1-ol), while the C 20 tetraenyl alcohol had low activity. Lower in vitro antiviral activity for saturated alcohols has been disclosed by Snipes et al., Ibid., 11, 98-104 (1977); and Snipes et al., Symp. Pharm. Lipid Effects (AOCS Monograph No. 5), 63-74 (1978).

There is still a need for an improved active agent that has antiviral, antibacterial and/or antifungal activity, such as a potent topical agent against viruses, bacteria, and/or fungi. For example, there is a need for an antiviral, antibacterial, antifungal agent in one agent that is active against enveloped virus, antibacterial and antifungal and that has very low toxicity, especially one that is a potent topical agent against viruses, bacteria, and fungi.

### SUMMARY OF EXEMPLARY EMBODIMENTS

In an aspect, the present disclosure provides a method for inactivating enveloped virus, bacteria, and/or fungi using agents with low cytotoxicity to humans and animals.

In an aspect, the present disclosure provides a composition comprising an active agent that has an anti-viral, antibacterial, and/or antifungal activity for topical application, which is effective for treating, preventing and/or reducing the lesions accompanying viral, bacterial, and/or fungal infections in animals and humans.

In an exemplary embodiment of the present disclosure, it is possible to provide a pharmaceutical composition for use in the method of the present disclosure.

Upon further study of the description and the appended claims, additional advantages of the compositions and methods of the exemplary embodiments of the present disclosure will become apparent to those skilled in the art.

Further, these and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a graph illustrating mean BW (g) of broilers from day of hatch to day 41 in chicks reared under the different additive treatments according to exemplary embodiment(s) of the present disclosure;

FIG. 2 is a graph of an exemplary mean daily growth rate (g/day) of broilers from day of hatch to day 41 in chicks reared under the different additive treatments according to exemplary embodiment(s) of the present disclosure; and

FIG. 3 is a graph of an exemplary distribution of dead (red) and alive, 41 day old broilers in the different treatments according to exemplary embodiment(s) of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

Cooper, sulfur and unsaturated fatty acids have antimicrobial and antiviral properties. Copper may be a natural antimicrobial and antiviral material. Ancient civilizations exploited the antimicrobial properties of copper long before the concept of microbes became understood in the nineteenth century. In addition to several copper medicinal preparations, it was also observed centuries ago that water contained in copper vessels or transported in copper conveyance systems was of better quality (i.e., no or little visible slime or biofouling formation) than water contained or transported in other materials.

The antimicrobial and antiviral properties of copper are still under active investigation.

### Exemplary Mechanisms of Antimicrobial Action of Copper

The oligodynamic effect was discovered in 1893 as a toxic effect of metal ions on living cells, algae, molds, spores, fungi, viruses, prokaryotic, and eukaryotic microorganisms, even in relatively low concentrations. This antimicrobial effect is shown by ions of copper as well as mercury, silver, iron, lead, zinc, bismuth, gold, and aluminum.

In 1973, researchers at Battelle Columbus Laboratories demonstrated that copper, in very small quantities, has the power to control a wide range of molds, fungi, algae and harmful microbes. Copper inhibits Actinomucor elegans, Aspergillus niger, Bacterium linens, Bacillus megaterium, Bacillus subtilis, Brevibacterium erythrogenes, Candida utilis, Penicillium chrysogenum, Rhizopus niveus, Saccharomyces mandshuricus, and Saccharomyces cerevisiae in concentrations above 10 g/L.

Candida utilis (formerly, Torulopsis utilis) is completely inhibited at 0.04 g/L copper concentrations.

Tubercle bacillus is inhibited by copper as simple cations or complex anions in concentrations from 0.02 to 0.2 g/L.

Achromobacter fischeri and Photobacterium phosphoreum growth is inhibited by metallic copper.

Paramecium caudatum cell division is reduced by copper plates placed on Petri dish covers containing infusoria and nutrient media.

Poliovirus is inactivated within 10 minutes of exposure to copper with ascorbic acid. Probeding some of copper's antimicrobial mechanisms and described no fewer than 120 investigations into the efficacy of copper's action on microbes. The antimicrobial mechanisms are very complex and take place in many ways, both inside cells and in the interstitial spaces between cells.

Examples of some of the molecular mechanisms noted by various researchers include the following:
The 3-dimensional structure of proteins can be altered by copper, so that the proteins can no longer perform their normal functions. The result is inactivation of bacteria or viruses. Copper complexes form radicals that inactivate viruses.
Copper may disrupt enzyme structures, and functions by binding to sulfur- or carboxylate containing groups and amino groups of proteins.
Copper may interfere with other essential elements, such as zinc and iron.
Copper facilitates deleterious activity in superoxide radicals. Repeated redox reactions on site specific macromolecules generate HO. radicals, thereby causing “multiple hit damage” at target sites.

Copper can interact with lipids, causing their peroxidation and opening holes in the cell membranes, thereby compromising the integrity of cells. This can cause leakage of essential solutes, which in turn, can have a desiccating effect.

Copper damages the respiratory chain in Escherichia coli cells. and is associated with impaired cellular metabolism.

Faster corrosion correlates with faster inactivation of microorganisms. This may be due to increased availability of cupric ion, Cu2+, which is believed to be responsible for the antimicrobial action.

In inactivation experiments on the flu strain, H1N1, which is nearly identical to the H5N1 avian strain and the 2009 H1N1 (swine flu) strain, researchers hypothesized that copper's antimicrobial action probably attacks the overall structure of the virus and therefore has a broad-spectrum effect.

Microbes require copper-containing enzymes to drive certain vital chemical reactions. Excess copper, however, can affect proteins and enzymes in microbes, thereby inhibiting their activities. Researchers believe that excess copper has the potential to disrupt cell function both inside cells and in the interstitial spaces between cells, probably acting on the cells' outer envelope.

Currently, researchers believe that the most important antimicrobial mechanisms for copper are as follows:
Elevated copper levels inside a cell causes oxidative stress and the generation of hydrogen peroxide. Under these conditions, copper participates in the so-called Fenton-type reaction—a chemical reaction causing oxidative damage to cells.
Excess copper causes a decline in the membrane integrity of microbes, leading to leakage of specific essential cell nutrients, such as potassium and glutamate. This leads to desiccation and subsequent cell death.

While copper is needed for many protein functions, in an excess situation (as on a copper sulfide surface), copper binds to proteins that do not require copper for their function. This “inappropriate” binding leads to loss-of-function of the protein, and/or breakdown of the protein into nonfunctional portions.

There is evidence today regarding copper's efficacy to destroy E. coli O157:H7, methicillin-resistant Staphylococcus aureus (MRSA), Staphylococcus, Clostridium difficile, influenza A virus, adenovirus, and fungi.

MRSA

Methicillin-resistant Staphylococcus aureus (MRSA) is a dangerous bacteria strain because it is resistant to beta-lactam antibiotics. Recent strains of the bacteria, EMRSA-15 and EMRSA-16, are highly transmissible and durable. This is of extreme importance to those concerned with reducing the incidence of hospital-acquired MRSA infections.

In 2008, after evaluating a wide body of research mandated specifically by the United States Environmental Protection Agency (EPA), registration approvals were granted by EPA in 2008 granting that copper kill more than 99.9% of MRSA within two hours.

Subsequent research conducted at the University of Southampton (UK) compared the antimicrobial efficacies of copper products to kill MRSA.

In 2004, the University of Southampton research team was the first to clearly demonstrate that copper inhibits MRSA.

Influenza a Virus

Influenza, commonly known as flu, is an infectious disease from a viral pathogen different from the one that produces the common cold. Symptoms of influenza, which are much more severe than the common cold, include fever, sore throat, muscle pains, severe headache, coughing, weakness and general discomfort. Influenza can cause pneumonia, which can be fatal, particularly in young children and the elderly.

After incubation for one hour on copper, active influenza A virus particles were reduced by 75%. After six hours, the particles were reduced on copper by 99.999%.

Once surfaces are contaminated with virus particles, fingers can transfer particles to up to seven other clean surfaces. Because of copper's ability to destroy influenza A virus particles, copper can help to prevent cross-contamination of this viral pathogen.

Adenovirus

Adenovirus is a group of viruses that infect the tissue lining membranes of the respiratory and urinary tracts, eyes, and intestines. Adenoviruses account for about 10% of acute respiratory infections in children. [© 1995-2020 The Nemours Foundation/KidsHealth, https://www.rchsd.org/health-articles/adenovirus/] These viruses are a frequent cause of diarrhea.

In a recent study, 75% of adenovirus particles were inactivated on copper within one hour. Within six hours, 99.999% of the adenovirus particles were inactivated.

An exemplary composition comprising copper can be a substitute for antibiotics, antivirals, and/or antifungal drugs, and as a means to treat or prevent infections with viruses, bacteria and/or fungi.

Combination treatment with antibiotics and a composition according to exemplary embodiments of the present disclosure may be recommended in patients with coronavirus.

The product has a wide range of applications in fields including medicine, veterinary medicine, agriculture, hygiene, etc. products.

### For the Treatment of People, Animals, Plants

The compounds, compositions and methods of the present disclosure can be useful for the treatment, alleviation or prevention of a wide range of viral, bacterial, and/or fungal diseases, including treatment or prevention of the COVID-19 virus. The compounds, compositions and methods of the present disclosure can be useful for the treatment, alleviation or prevention of a number of diseases and conditions, including abnormal sugar levels in blood, abnormal cholesterol levels in blood, AIDS, cold, epilepsy or other neurological diseases and conditions, schizophrenia, and arthritis.

Application to the nose of the compounds or compositions according to various exemplary embodiments of the present disclosure may make the environment denser and make it more difficult for a pathogen such as a virus to enter cells even if it enters into the nose. The epithelia cells in the nose is replaced and expunged every 15 minutes. A pathogen such as a virus can only develop past entry into the nose. Thus, a film is provided in the nose that prevents the entry of a pathogen such as a virus past nose and the pathogen may get exhaled. A similar effect may be achieved where the compounds or compositions according to various exemplary embodiments of the present disclosure are applied to the lips.

There are several nutritional supplements currently on the market that are produced by various nutritional supplements with additives containing various copper elements.

The compositions of exemplary embodiments according to the present disclosure differ from other food additives on the market in molecular level and structure level, a difference that affects the absorption of a substance into body, compared to other food supplements that are available in, e.g., capsule form (absorption of which in not as effective compared to the compositions of the present disclosure).

In an exemplary embodiment of the present disclosure, the composition may contain particles of a copper sulfide in a membrane comprising at least one substance that promotes absorption and penetration into cells, such as a lipid. These particles may be insoluble in water, and their size may be tiny—for example, about 30-40 nanometers (for comparison, the particle diameter of the coronavirus is about 125 nanometers). Copper has very strong viral properties, but it does not interact and does not use biological agents in intracellular fluid.

The compositions and methods of certain exemplary embodiments according to the present disclosure can be useful in the treatment or prevention of a wide range of viral, bacterial, and/or fungal diseases, by eliminating inflammation in soft tissues; this is due to the acceleration of the formation of granules tissue, slowing down the formation of scars during the inflammatory process.

Due to the minimum size of the constituent particles, they penetrate into the intracellular space, perform a viral action, and then are completely removed from the cell.

The presence of the minerals copper and sulfur in the human body is necessary for normal functioning the immune system and various systems of the body. High doses of copper in the body can cause diseases, such as hepatic and renal failure, and therefore it is necessary to control the dose.

Among other things, copper and sulfur help prevent inflammation, which can catalyze enzyme superoxide dismutase (SOD), an enzyme that is a powerful antioxidant that helps protect free radicals. In addition, copper is a catalyst for histaminase, which breaks down histaminase, which is responsible for the reaction the body to allergenic substances.

The particles of the compounds or compositions of certain exemplary embodiments according to the present disclosure have the smallest sizes of its parts, which allows maximum absorption, providing the minimum amount of material for effective action.

The solutions currently offered for combating the corona virus are aimed at disinfecting surfaces, isolating patients and people exposed to the virus at different levels and providing instructions for daily behavior and increased hygiene. These solutions are good and practical, but as we can see every day is not quite suitable for a practical test due to people's lifestyle and character modern world.

The compounds and compositions of certain exemplary embodiments according to the present disclosure offer a solution to treat, suppress, or prevent the development of viruses in the human body and treat, suppress or prevent infections. The product can provide protection and support to the human body in a state with exposure to the corona virus and treatment of the effects of inflammation and scarring.

While the existing treatment and prevention methods offer a solution focused on the human environment (isolation and cleaning as prevention of infections), the compositions and methods of the present disclosure focus on the human body by, for example, strengthening immune system as a means of fighting the virus and preventing infection.

The compounds and compositions of the present disclosure have been tested on animals and have shown positive results in improving immune function and suppression of the growth of viruses and bacteria. In laboratory experiments on birds, pigs and fish, in different doses of the active agent(s) and other environmental conditions, delays could be seen in the development of viruses and a significant decrease in the amount of provided active agent(s), compared to controls where no connection was provided. These results are especially important in light of the fact that some populations have been exposed to harsh conditions that should have made their recovery more difficult than the deadly virus, but they survived.

In the experiments, the viruses administered are the V-H strain of Newcastle virus Newcastle virus refers to family Paranoxyviridae, which, like viruses belonging to the family Coronaviridae, is an RNA chain monovirus responsible for respiratory and digestive tract infections among mammals and humans.

In addition, the toxicity of the high dose composition(s) was tested, and it was also found that the high dose composition(s) did not exhibit copper poisoning.

Experiments on birds and fish, in which hundreds of animals participated, were carried out. Experiments on 1500 pigs were carried out. In Peggy Pig, the positive results are significant, as the respiratory system in these animals is underdeveloped and extremely vulnerable to viral damage. 80% of pigs returned to the herd, survived and recovered, instead of going to slaughter after treatment.

Another experiment, against infecting dozens of aggressive birds with E-Coli. The test results showed relatively moderate mortality—24%. The mortality in the control group was 100%.

### Exemplary Compositions and Uses to Stop or Prevent Virus Outbreaks Over Time

A nasal spray or oral administration may be most effective and is preferred. This may allow quick action and may be very affordable.

C 20-24 linear polyunsaturated acids suitable for use in the present methods and compositions include eicosapentaenoic acid (EPA), eicosahexaenoic acid, eicosaheptaenoic acid, heneicosapentaneoic acid, heneicosehexaenoic acid, heneicoseheptoceinic acid A, docosaheptaenoic acid, tricosapentaenoic acid, tricosahexaenoic acid, tricosaheptaenoic acid, tetracosapentaenoic acid, tetracosahexaenoic acid, tetracosaheptaenoic acid, alpha-eleostearic acid, and mixtures thereof. Preferred acids include eicosapentaenoic acid (EPA), docosahexaenoic acid (DHA), and mixtures thereof, including both unconjugated and conjugated double bond isomers. 5,8,11,14,17-EPA and 4,7,10,13,16,19-DHA and mixtures thereof are particularly preferred. It should be understood that the above polyunsaturated acids may exist as a variety of geometric isomers, all of which are included in the invention.

Polyunsaturated acids may be used in the form of pure compounds or mixtures of pure compounds, or they may be used in the form of concentrates obtained from natural plant and/or animal sources. Natural sources of polyunsaturated acids suitable for use in the invention include fish liver oils and their concentrates and/or extracts. It is known that EPA, DHA, and alpha-eleostearic acid are present in significant amounts in oils such as cod liver oil, halibut liver oil, tuna liver oil and bitter melon oil, tung oil and the like. Saponification and/or solvent extraction of cod liver oils, halibut liver oil, tuna liver oil and bitter melon oil, tung oil, and the like can increase the percentage of free polyunsaturated fatty acids available from them, which percentage may be further increased by subsequent concentration and/or additional extraction.

C 20-24 linear polyunsaturated acids, which are not available from natural sources, may be synthesized by conventional methods to obtain long-chain polyolefins containing cis or trans double bonds. Such olefin syntheses are commonly disclosed in the olefin synthesis chapters in, e.g., Harrison et al., “Compendium of Organic Synthetic Methods” (Wiley 1971); and Carruthers, Some Contemporary Methods of Organic Synthesis (Cambridge, 1971); and with respect to closely related carotene systems in Anand et al., “Art in Organic Synthesis” (Holden-Day 1970). The foregoing is illustrative and does not include all such general references. The carboxyl group may be introduced at an early stage, as a protected, e.g., esterified, function, or at the end of the synthetic route by conventional means, as shown in the above references. Longer chain acids may also be obtained by homologating acids with fewer carbon chain atoms using conventional reaction sequences, for example, Arndt-Eister homologation and the like.

The various geometric and positional isomers of polyunsaturated acids and/or their associated alcohols and aldehydes may be obtained by one or more standard separation methods well known in the art, such as column chromatography, thin layer chromatography, headspace chromatography, high performance liquid chromatography, fractional crystallization, and the like. Partial separations such as solvent extraction, molecular distillation to obtain higher active fractions are also included in separation methods for preparing the agents for use in the present processes and compositions.

The polyunsaturated acids of the present disclosure may be administered in the form of pharmaceutically acceptable inorganic or organic base addition salts, wherein the salts have comparable and/or other beneficial virucidal activity and which are otherwise physiologically compatible. Suitable inorganic bases for the formation of these salts include, for example, hydroxides, carbonates, bicarbonates and alkoxides of alkali metals or alkaline earth metals such as sodium, potassium, magnesium, calcium and selenium, and also a copper sulfide, and the like. Suitable organic bases include the following amines: lower mono-, di- and tri-alkylamines, the alkyl moieties of which contain up to three carbon atoms, for example, methylamine, dimethylamine, trimethylamine, ethylamine, di- and triethylamine, N-methyl-N-ethylamine and the like; mono-, di- and tri-alkanolamines, the alkano-moieties of which contain up to three carbon atoms, for example, mono-, di- and tri-ethanolamine, alkylenediamines containing up to six carbon atoms, for example hexamethylenediamine; phenylalkylamines such as benzylamine, phenylethylamine and N-methylphenylethylamine; cyclic saturated or unsaturated bases containing up to six carbon atoms, for example pyrrolidine, piperidine, morpholine, piperazine and their N-alkyl and N-hydroxyalkyl derivatives, such as N-methylmorpholine and N-(2-hydroxyethyl) piperidine, as well as pyridine.

The compositions of various exemplary embodiments according to the present disclosure may include the following active ingredients: copper sulfide micro elements in the form of nanoparticles, linoleic acid (polyunsaturated omega-6 fatty acids), oleic acid (monounsaturated omega-9 fatty acid), alpha-eleostearic acid, and kenaf oil.

The antiviral, antibacterial, and/or antifungal properties of the compounds and compositions of various exemplary embodiments according to the present disclosure suggest their use to prevent the spread of infection, for example, by incorporating them into a hand cream or lotion for use by physicians, both before and after examining patients with suspected infections. In addition, the compounds and compositions of various exemplary embodiments according to the present disclosure may be used in fluids used to kill viruses for examining tables, instruments, gloves, towels and other surfaces that may come in contact with patients or physicians during medical examinations. The low toxicity of the compounds and compositions of various exemplary embodiments according to the present disclosure further enhances their attractiveness for such prophylactic use.

The exemplary compounds of various exemplary embodiments according to the present disclosure may be used in admixture with conventional excipients, i.e., pharmaceutically acceptable organic or inorganic carrier substances, suitable for parenteral or enteral administration, which do not enter into a harmful, degrading, or destructive reaction with the active compounds. Suitable pharmaceutically acceptable carriers include, but are not limited to, water, saline solutions, alcohols, vegetable oils, polyethylene glycols, gelatin, lactose, amylose, magnesium stearate, talc, silicic acid, viscous paraffin, perfume oil, monoglycerides and diglycerides of fatty acids. fatty acid esters, hydroxymethylcellulose, polyvinylpyrrolidone, and the like. Pharmaceutical preparations may be sterilized and, if desired, may be mixed with auxiliary agents, for example, lubricants, preservatives, stabilizers, wetting agents, emulsifiers, salts for influencing osmotic pressure, buffers, dyes, flavors and/or aromas, etc., that do not have a harmful effect on or a harmful reaction with the active compounds.

For parenteral administration, solutions, preferably oily or aqueous solutions, as well as suspensions, emulsions or implants, including suppositories, are particularly suitable. Ampoules are convenient unit dosages.

Particularly suitable for enteral administration include tablets, dragees, or capsules, which may contain talc and/or a carbohydrate carrier, or a binder, or the like. The carrier preferably includes lactose and/or corn starch and/or wheat starch and/or potato starch. Also suitable are a syrup, elixir, and the like, which use a sweetened carrier. Sustained release compositions may be formulated, including those in which the active compound(s) is protected by differentially degradable coatings, e.g., microencapsulation, multiple coatings, and the like.

Exemplary antiviral, antibacterial, and/or antifungal compositions of various exemplary embodiments according to the present disclosure may be used as a topical composition, either in a non-nebulized or nebulized form. Non-sprayable forms include semi-solid or solid forms, including a carrier typical for topical application, and having a dynamic viscosity, preferably greater than water. Suitable formulations include, but are not limited to, solutions, suspensions, emulsions, creams, ointments, powders, ointments, ointments, and the like. If desired, they may be sterilized or mixed with auxiliary agents such as preservatives, stabilizers, wetting agents, buffers or salts to influence osmotic pressure, and the like. Preferred carriers for non-spray topical formulations include ointment bases, for example, polyethylene glycol-1000 (PEG-1000); conventional ophthalmic devices; creams such as HEB cream; and gels such as K-Y gel; as well as petroleum jelly and the like. The topical preparations may also contain emollients, fragrances and/or pigments to increase their acceptability for various uses.

Also suitable for topical use are aerosol sprays in which the antiviral, antibacterial, and/or antifungal compound(s), preferably in combination with a solid or liquid inert carrier, is packaged in a squeeze bottle or mixed with a volatile, usually gaseous, pressurized propellant such as freon (chlorofluorocarbon), or an environmentally friendly volatile propellant. Such compositions may be used for application to environmental surfaces such as examination tables, toilet seats, and the like, and/or for application to skin or mucous membranes. Aerosol or aerosol preparations may contain solvents, buffers, surfactants, fragrances and/or antioxidants in addition to the antiviral, antibacterial, and/or antifungal compound(s) of various exemplary embodiments according to the present disclosure.

For preferred topical application, especially for the treatment of humans and animals suffering from symptoms of viral, bacterial, and/or fungal infections, it is preferred to use the polyunsaturated acids of the present disclosure, although unsaturated alcohols and aldehydes are also suitable. Acid salts may be less effective for topical application. It should be understood that salts may be used to prepare topical compositions in combination with suitable buffers and/or acids to lower the pH of the final formulation.

Antiviral, antibacterial, and/or antifungal compositions of the exemplary embodiments according to the present disclosure may be administered to animals, especially mammals, containing at least one antiviral, antibacterial, and/or antifungal compound in an effective amount and in unit dosage form. The dose may be administered separately or in divided doses throughout the day.

In a preferred topical application form used to implement the present method, an antiviral, antibacterial, and/or antifungal composition comprising an effective amount of an antiviral, antibacterial, and/or antifungal compound of various exemplary embodiments according to the present disclosure may be administered to an infected area, such as the surface of the skin, mucous membranes, of an animal or human suffering from a viral, bacterial and/or fungal infections, in a dose that ranges from about 0.001 mg to about 1 g per application. The dose amount administered may be adjusted depend on factors such as the area to be treated, the severity of the symptoms and the nature of the antiviral, antibacterial, and/or antifungal agent and topical carrier used. It is preferable to use dosages in the range of 0.01 mg to 100 mg. A preferred topical formulation is an ointment that uses about 0.01 mg to 50 mg of the antiviral, antibacterial, and/or antifungal agent per cc of the ointment base, the latter preferably being PEG-1000, and more preferably, an ointment containing about 0.1 mg to 10 mg C 20-24 polyunsaturated fatty acids, preferably DHA and/or EPA, per cubic centimeter ointment base, preferably PEG-1000.

In the preparation of antiviral, antibacterial, and/or antifungal compositions according to the present disclosure, in particular preparations for topical use comprising polyunsaturated acids, a compound containing copper and sulfur, such as a copper sulfide, it is preferable to use polyunsaturated fatty acids that are as pure as possible and/or that essentially do not contain or have at least a significantly reduced content of esters, e.g., triglycerides. Thus, if fish oil is used as a source of polyunsaturated acids, it will be beneficial to reduce the content of natural triglycerides, for example, by saponifying the oil and recovering the saponified acid fraction.

Pharmaceutical formulations that use substantially pure polyunsaturated C 20-24 fatty acids having 5-7 double bonds are preferred, especially those that are substantially free of esters such as triglycerides, and most preferably those that contain substantially pure EPA and/or DHA, as essentially the only fatty acids in it. When a concentrate of polyunsaturated acids is used to prepare a pharmaceutical composition, the content of C 20-24 fatty acids having 5-7 double bonds relative to the final product as 100% by weight is preferably at least about 20% by weight, more preferably at least about 30%, at least about 40%, at least 50%, at least 60%, at least 70%, at least 80%, at least 90%, at least 95% or at least 99%.

The content of copper in the compositions of the present disclosure may be in a range of 0.03% to 3% by mass relative to the total amount of the composition as 100% by mass.

The content of sulfur in the compositions of the present disclosure may be in a range of 0.1% to 10% by mass relative to the total amount of the composition as 100% by mass.

It should be understood that the formulations and dosages may vary and may be outside the preferred ranges for various applications, for example, application to environmental surfaces for prophylactic use and/or for veterinary and disinfection applications.

Aerosols for topical use in medicine may have the same concentrations and dosages as the creams, lotions and ointments described above, but may have higher or lower concentrations for other applications, for example, for prophylactic and/or disinfecting applications, veterinary applications etc.

Dosage levels for enteral and/or parenteral administration to achieve systemic activity may be typically in the range of 0.001 mg to 5 g per day, preferably in solid or liquid unit doses of about 0.01 mg to 1 g of antiviral, antibacterial and/or antifungal compositions, together with about 0.1 g to 10 g of a pharmaceutically acceptable carrier. The exact dosages and frequency of administration may vary depending on factors such as the severity of the clinical symptoms and the nature of the antiviral, antibacterial, and/or antifungal compositions and the virus species being treated, as well as the nature and size of the patient in a manner well known to veterinarians and medical practitioners.

An exemplary method for making the compositions according to various exemplary embodiments of the present disclosure may comprise: providing a mixture of a copper sulfide, tung oil, and kenaf oil; heating the mixture at about 240° C. to obtain a heated mixture; and cooling the heated mixture. In the heated mixture, the hydrocarbon chains of the oils break and the copper sulfide enters into the oils.

It is believed that, without further elaboration, one skilled in the art can, using the previous description, use the present invention to its fullest extent. Therefore, the following preferred specific embodiments are to be construed as merely illustrative, and not limiting the remainder of the disclosure in any way.

### EXAMPLE(S)

An example of a composition according to various exemplary embodiments of the present disclosure includes the following active ingredients: copper sulfide micro elements in the form of nanoparticles, linoleic acid (polyunsaturated omega-6 fatty acids), oleic acid (monounsaturated omega-9 fatty acid), alpha-eleostearic acid, and kenaf oil.

Biological Qualities

The active ingredients of the administered composition are the copper sulfide in nanoparticles that form a complex with the following unsaturated fatty acids: linoleic acid (polyunsaturated omega-6 fatty acid) and oleic acid (monounsaturated omega-9 fatty acid). The combination of the copper sulfide nanoparticles and the unsaturated fatty acids are characterized by high bioavailability and an effective stimulating effect on the body.

The mechanism of action of the composition is due to the properties active ingredients-trace elements of the copper sulfide, and polyunsaturated linoleic and oleic fatty acids, and alpha-eleostearic acid. Copper sulfides are indispensable, vital microelements that play a regulating role in protein, fat, carbohydrate and mineral metabolism in animals organisms. It is found in all tissues of the body. This is part of the enzyme content-biological catalysts. It participates in the regulation of redox and neuroendocrine processes and lipid peroxidation, while simultaneously acting as an antioxidant. Copper sulfides are components in metalloproteins that control immunogenesis and cellular respiration, transport and absorption of oxygen and molecular nitrogen in tissues and organs. They are involved in hematopoiesisin the presence of iron, in the creation of hemoglobin in the blood. A copper sulfide regulates the absorption of iron in the gastrointestinal tract and participate in its transformation into an accessible hematoform and its transport to the bone marrow. Copper is involved in the creation of bones and connective tissue and affects the healthy development of the fetus. Copper contributes to the normalization of the metabolism of vitamins of group B, A, C, and group P. Copper sulfides contribute to the development of nonspecific resistance of human and animal organisms, active growth and development of youth in humans and animals, decrease in morbidity and increased survival, prevention of alimentary anemia, osteomalacia, rickets, cardiovascular and central nervous system disorders.

Linoleic acid (CH3 (CH2) 3-(CH2CH═CH) 2 (CH2) 7COOH) is a monobasic carbonic acid. It is an omega-6 polyunsaturated fatty acid. It is irreplaceable in bodily development. It is not created in animal organisms and is not ingested with food. When ingested in a healthy body, it quickly separates into arachidonic and gamma-linoleic acids.

Arachidonic acid is an antioxidant that is directly involved in the regulation of processes, protection of blood vessels, maintenance of elasticity and density of their walls, membrane-protective processes in cells, regulation of the functions of the adrenal glands and gonads, blood and regulation of lymph flow, increasing the level of immune defense, neutralizing metabolic inflammatory processes, accelerating tissue processes regeneration and improvement of metabolism of cells, tissues and organs.

Gamma-linoleic acid is involved in the synthesis of prostaglandins, regulation of the production of hydrochloric acid in the gastrointestinal tract, increased production of protective gastric mucus, growth and regeneration of muscle elements, has an anti-inflammatory and coagulation-improving effect, is indispensable for the growth and development of animals. Linoleic and arachidonic acids are indispensable for the normal functioning of cell membranes; metabolic products of arachidonic acid formed in mast cells and other cells are involved in local inflammatory reactions that cause the removal of pathogens. Polyunsaturated fatty acids are part of the F vitamins.

Oleic acid (CH3 (CH2) 7CH═CH (CH2) 7COOH) is a monounsaturated carbonic acid. It is a monounsaturated omega-9 fatty acid. It plays a large role in animal nutrition. It improves the absorption of nutrients, serves as a source of cellular energy and is part of the lipids involved in cell membrane construction. It optimizes a healthy digestive system composition of lipids taken from food and increases metabolic energy consumption.

Exemplary Quantities of the Composition.

The composition does not cause any harmful side effects. It is classified as a low hazard class 4 in terms of its toxicity to humans and animals. Active in wide temperature range in water of any hardness, resistant to pH changes.

### AVIR Forte

The objective of the current preliminary study was to evaluate the effects of AVIR Forte addition to the water at different concentration on the growth rate and sustainability of broilers up until they reach the age of poultry marketing.

### Exemplary Materials and Methods

Contents of Fatty Acids in the Product Avir Forte
(Analysis 12.03.18)

| | | | |
|---|---|---|---|
| C10 | 2 | g/L | |
| C12 | 0.1 | g/L | |
| C14 | 0.6 | g/L | |
| C16 | 54.5 | g/L | |
| C18.0 | 17.8 | g/L | |
| C18.1 | 407.5 | g/L | (oleic) |
| C18-2 | 1.85 | g/L | (Linole) |
| C20 | 12.35 | g/L | |

Experimental Design

All the procedures in this study were carried out in accordance with the accepted ethical and welfare standards of the Israel Ethics Committee (permission # IL-780/18). AVIR Forte: the additive was given to the chicken in the form of liquid.

A total of 260 day-old male broiler chicks, from Ross 308 strain, were obtained from a breeder flock of hens, during their optimal period of egg production (33-wk-old). The chicks were individually weighed, 220 chicks with a body weight (BW) of 37±6 g were selected. Each chick was individually tagged.

The 220 chicks were divided according to their BW into 4 treatment groups (n=55 per group) with similar starting weight. All 55 chicks were kept at the same pen, on pine wood shaving floor beading.

The treatment groups included: 1. Control, 2. Concentration I 0.25 ml AVIR Forte/1 Kg body weight, 3. Concentration II 0.5 ml AVIR Forte/1 Kg body weight and 4. Concentration III 1 ml AR-DEO/1 Kg body weight.

On day 12, all chicks were vaccinated against infected bronchi (VIR 111 batch no 1-031982). At day 16 AVIR Forte was administrated to the chick's drinking water for three consecutive days according to the above treatments. At day 27 a second administration of AVIR Forte to the chick's drinking water was done for three consecutive days according to the treatments.

Water and feed, were available for ad-libitum consumption. The standard diets were designed to meet the breeder recommendations. The birds were held under a lights regime of 18L:6D h. Each chick was weighed every week and pen weekly food intake was calculated.

Statistics

All data were subjected to statistical analysis using one-way Analysis of Variance (ANOVA). Values that differed (at a level of p<0.05) were considered statistically significant. In addition, Tukey-Kramer test was conducted comparing the treatment averages.

### Exemplary Results

Body Weight and Growth Rate:

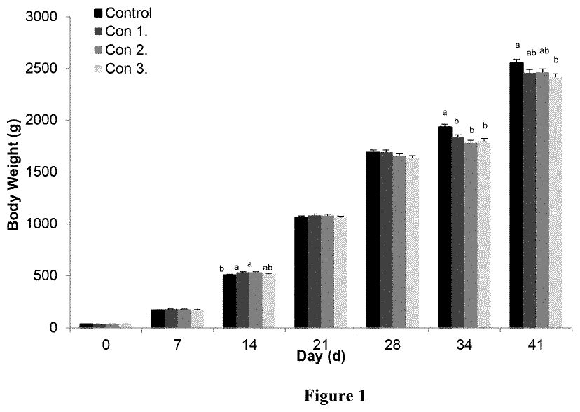

The growth of the hatchlings up to age of 41d, clearly indicates that the dietary treatments had a significant effects on broilers BW (FIG. 1). The chicks that drank AVIR Forte concentration III (1 ml AR-DEO/1 Kg body weigh) were found to have significantly lower weight compared to control chicks on d34 and d41.

Mean body weights of broilers from the AVIR Forte concentration II and I were found also to be significantly lower compare to broilers from the control group at d34. At d41 their bw remained lower than control however the difference was not significant.

All treatments had a similar growth rate during the brooding period (d0 to 21d). From day 21 Up to day 34 daily ambient temperatures had a major impact on broiler performance and their growth rate was affected. During that period daily ambient temperature was above 35° C. due to severe heat waves, while growth rate of AVIR Forte, treatments was ±20 g/d control broilers grew±40 g/d.

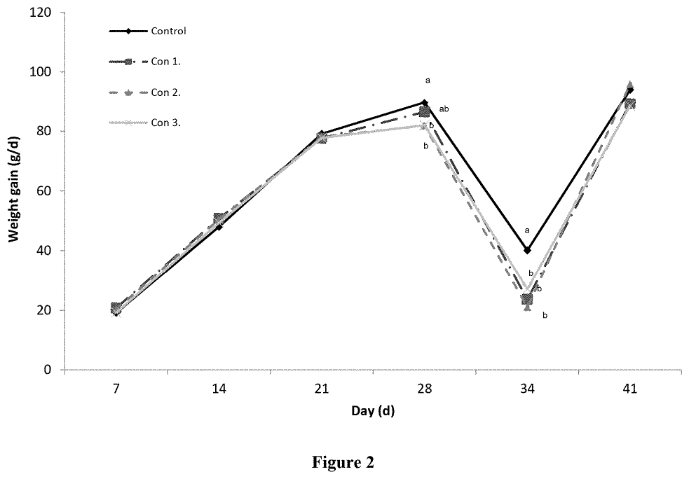

During the sixth week, ambient temperature had stabilized and were less than 28° C. during the day. Those condition enabled the broiler growth to reciprocate and growth rate was ±90 g/d in all four treatments group (FIG. 2).

Mortality and Sustainability:

During the growing period two stressful factors had affected the broilers sustainability.

The first factor was the IBV vaccine that was administrated at the age of 12 day. Following the administration of the vaccine an immune response of the respiratory system was identified; all chicks were found to wheeze in all 4 treatments. The wheezing had stop during the third week and it had no effect on the livability of the broilers.

The second factor was ambient temperature-specifically severe heat waves that occurred during week four and five, and at the last two days of experiment day 40-41. while ambient temperature was found to effect broilers performance during week four and five, it had no effect on the livability of the broilers. However the last fatal heat wave with room temperature reaching above 40° C. on days 40-41 had claimed the life of 40% of the control broilers and 22 (out of 55) broilers had died during those fatal 24 h. Only one broiler in each of the AVIR Forte I and II treatment had died during the final heat wave (Table 1)

TABLE 1
Mortality and livability of broilers in all four treatment groups.

| | | alive | Dead |
|---|---|---|---|
| Control | (n) | 33 | 22 |
| | (%) | 60.00 | 40.00 |
| Con. 1 (AVIR Forte I) | (n) | 54 | 1 |
| | (%) | 98.18 | 1.82 |
| Con. 2 (AVIR Forte II) | (n) | 54 | 1 |
| | (%) | 98.18 | 1.82 |
| Con. 3 (AVIR Fort III) | (n) | 55 | 0 |
| | (%) | 100 | 0 |

The difference in mortality rate between control and the AVIR Forte groups was found to be highly significant with a Pearson test probability of P (χ²)<0.0001

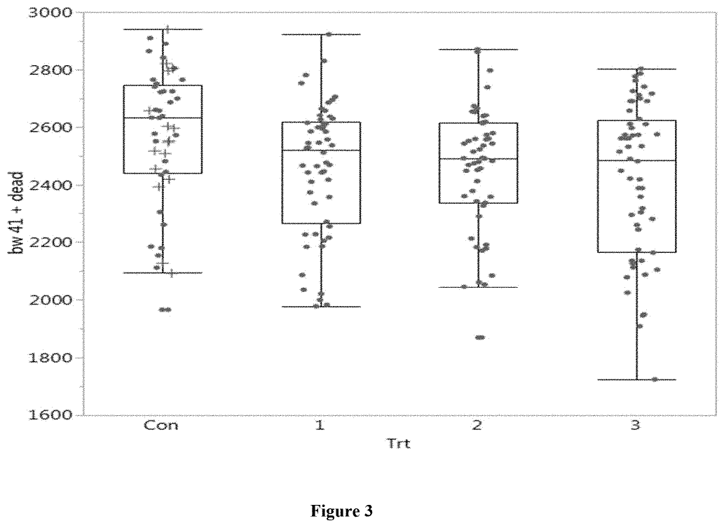

Distribution of day 41 bw of dead and alive birds is displayed in FIG. 3. Although control broilers had a mean heavier bw in comparison to the AVIR Forte treatment broilers, dead birds bw vary from 2100 g up to 2800 g. Meaning not just the heaviest broilers in the control treatment died, while broilers with similar bw from all three AVIR Forte treatments prevailed.

Feed Intake and Feed Conversion Rate (FCR):

Data provided in Table 2, represent group mean weekly and total FCR from day of hatch to d41 (n=1).

Although all broilers grew less during the fifth week, feed consumed was high, and FCR was affected. Due to the fact that control broilers growth was higher their FCR was lower compared with the rest of the treatments. However, calculation of FCR for the entire growing period indicates that control FCR is the higher and with the increase in AVIR Forte concentration there is a positive tendency in FCR reduction.

Nevertheless one should remember that there is only one replicate per treatment group and the results are not significant.

TABLE 2
Feed conversion rate (FCR) of each treatment
calculated per week and total for that all period (0 to 41 d).

| | FCR | FCR | FCR | FCR | FCR | FCR | FCR |
|---|---|---|---|---|---|---|---|
| | 1 wk | 2 wk | 3 wk | 4 wk | 5 wk | 6 wk | total |
| Control | 1.09 | 1.21 | 1.33 | 1.51 | 3.88 | 1.97 | 1.71 |
| Con. 1 (AVIR | 0.99 | 1.12 | 1.25 | 1.51 | 5.18 | 1.79 | 1.65 |
| Fort I) | | | | | | | |
| Con. 2 (AVIR | 1.01 | 1.18 | 1.26 | 1.53 | 7.64 | 1.67 | 1.67 |
| Fort II) | | | | | | | |
| Con. 3 (AVIR | 1.02 | 1.06 | 1.23 | 1.49 | 4.40 | 1.72 | 1.60 |
| Fort III) | | | | | | | |

### Further Exemplary Summary

In order to better understand the effects of the different AVIR Forte concentrations, the broilers were exposed to varying natural environmental condition. In the current experiment, we found a positive significant influence on broilers sustainability when exposed to hot environmental condition.

Although the AVIR Forte III concentration influenced broilers growth performance and they did not grow as well as the control, with a significant lower bw at day 41, total FCR of this treatment was the lowest (only 1 replicate).

In order to give proper recommendations to poultry farmers we suggest a series of experiments imitating different environmental condition-conducted on several pens per concentration in order to identify the true influence on feed consumption and FCR.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

What is claimed is:

1. A method for inactivating at least one of an enveloped virus, a bacterium or a fungus which comprises contacting said virus, bacterium, and fungus with an effective amount of a composition comprising:
a copper sulfide, and
at least one of alpha-eleostearic acid, a pharmaceutically acceptable salt of said alpha-eleostearic acid, C20-24 linear polyunsaturated fatty acid having 5-7 double bonds, a pharmaceutically acceptable salt of said C20-24 linear polyunsaturated fatty acid, C20-24 linear polyunsaturated aldehyde having 5-7 double bonds, C20-24 linear polyunsaturated primary alcohol having 5-7 double bonds, and a mixture thereof.

2. The method of claim 1, wherein the composition comprises the copper sulfide, and alpha-eleostearic acid, a C20-24 linear polyunsaturated acid having 5-7 double bonds, or a mixture of said acids.

3. The method of claim 2, wherein the composition comprises the copper sulfide, and alpha-eleostearic acid, eicosapentaenoic acid (EPA), docosahexaenoic acid (DHA), or a mixture thereof.

4. The method of claim 2, wherein the composition comprises the copper sulfide, 5,8,11,14,17-EPA, 4,7,10,13,16,19-DHA, and alpha-eleostearic acid.

5. The method of claim 4, where the composition is applied topically to reduce or inhibit lesions in an animal or a human.

6. A method of treating an infection in a subject caused by at least one of an enveloped virus, a bacterium, or a fungus, comprising administering to the subject:
a copper sulfide, and
an effective amount of at least one of alpha-eleostearic acid, a pharmaceutically acceptable salt of said alpha-eleostearic acid C20-24 linear polyunsaturated fatty acid having 5-7 double bonds, a pharmaceutically acceptable salt of said C20-24 linear polyunsaturated fatty acid, C20-24 linear polyunsaturated aldehyde having 5-7 double bonds, C20-24 linear polyunsaturated primary alcohol having 5-7 double bonds, and a mixture thereof.

7. The method of claim 6, wherein the composition comprises the copper sulfide, and alpha-eleostearic acid, a C20-24 linear polyunsaturated acid having 5-7 double bonds, or a mixture of said acids.

8. The method of claim 7, wherein the composition comprises the copper sulfide, and alpha-eleostearic acid, eicosapentaenoic acid (EPA), docosahexaenoic acid (DHA), or a mixture thereof.

9. The method of claim 7, wherein the composition comprises the copper sulfide, 5,8,11,14,17-EPA, 4,7,10,13,16,19-DHA, and alpha-eleostearic acid.

10. The method of claim 9, where the composition is applied topically to reduce or inhibit lesions in an animal or a human.

11. A composition for inactivating an enveloped virus, a bacterium, and/or a fungus, comprising:
a copper sulfide; and
at least one of alpha-eleostearic acid, a pharmaceutically acceptable salt of said alpha-eleostearic acid, C20-24 linear polyunsaturated fatty acid having 5-7 double bonds, a pharmaceutically acceptable salt of said C20-24 linear polyunsaturated fatty acid, C20-24 linear polyunsaturated aldehyde having 5-7 double bonds, C20-24 linear polyunsaturated primary alcohol having 5-7 double bonds, and a mixture thereof.
